(19) United States
(12) Patent Application Publication
(10) Pub. No.: US 2024/0207096 A1
(43) Pub. Date: Jun. 27, 2024

(54) ASSEMBLY OF A VITRECTOR NEEDLE

(71) Applicant: JOHNSON & JOHNSON SURGICAL VISION, INC., Irvine, CA (US)

(72) Inventors: Alexander Shechtman, Haifa (IL); Ilya Sitnitsky, Nahariya (IL); Stanislav Katzir, Hadera (IL); Elad Avraham Diukman, Haifa (IL); Assaf Govari, Haifa (IL)

(21) Appl. No.: 18/387,487

(22) Filed: Nov. 7, 2023

Related U.S. Application Data
(60) Provisional application No. 63/435,509, filed on Dec. 27, 2022.

(51) Int. Cl. A61F 9/007

(52) U.S. Cl. CPC A61F 9/00763

(57) ABSTRACT

A method for assembling a vitrector, including: providing a distal housing portion for the vitrector, a transmission unit driving a needle into oscillation, an assembly pin to be inserted into the transmission unit to fix the needle in position, and a needle casing, having a lumen, the casing enclosing the needle within the lumen. The transmission unit is fixed to the distal housing portion, and the assembly pin is inserted into the transmission unit to fix a distal tip of the needle at a most distal position of the oscillation. The transmission unit is inserted, with the assembly pin inserted, to a predetermined position in the distal housing portion. The casing is inserted into the distal housing portion so that the distal tip butts a termination of the casing. The casing is fixed to the distal housing portion, and the assembly pin is removed from the transmission unit.

### CROSS-REFERENCE TO RELATED APPLICATIONS

The present application claims priority to and the benefit of U.S. Provisional Application No. 63/435,509, filed on Dec. 27, 2022, which is hereby incorporated by reference in its entirety.

### FIELD OF THE DISCLOSURE

This disclosure relates generally to vitrectomy, and specifically to tools used for vitrectomy.

### BACKGROUND

Vitrectomy is surgery that removes vitreous humor from an eye, and the surgery is performed using a vitrector, which comprises a hollow needle through which the vitreous humor is aspirated. Following are summaries of references that may apply to vitrectors.

U.S. Patent Application 2020/0016001, to McDonell et al., describes a vitrectomy instrument which may include a forward needle stop and a cutter stop to reduce dimensional variation of a cutter of the vitrectomy instrument.

European Patent 2,793,717, to Underwood et al., describes vitrectomy probes having an adjustable cutting port size, and features are described for adjusting the size of the cutting port.

U.S. Patent Application 2021/0113379, to Chon et al., describes a vitrectomy probe with a light emitting, visibly discrete traceable probe cap. It is stated that the light emitting character of the probe cap may aid a surgeon in tracing the end of a needle of the probe during a vitrectomy procedure.

U.S. Pat. No. 7,103,947, to Sartor et al., describes a spacer that may be positioned between jaw members prior to an overmolding process. The spacer sets a fixed gap distance between the jaw members at closure.

### BRIEF DESCRIPTION OF THE DRAWINGS

The present disclosure will be understood from the following detailed description, taken in conjunction with the drawings in which:

FIGS. 1A, 1B, and 1C are schematic diagrams of a vitrector;

FIG. 2 is a cross-sectional view of a driving section of the vitrector;

FIGS. 3A, 3B, and 3C are schematic cross-sections of a needle within a casing of the vitrector, for different positions of the needle; and

FIG. 4 is a flowchart of steps performed in assembling the vitrector.

### DESCRIPTION OF EXAMPLES

### Overview

A vitrector is a surgical tool used for removing vitreous humor from an eye, and comprises a hollow cylindrical needle through which the vitreous humor is aspirated. The needle is enclosed in a hollow cylindrical tubular casing, closed at its distal end, and the casing has a window in its side, close to the distal end, via which the vitreous humor transfers to the needle. During operation of the vitrector, the needle is oscillated within the casing, and the oscillation causes the distal tip of the needle to cut the vitreous humor as the tip traverses the casing window; it is the cut portions of the vitreous humor that are aspirated.

There are a number of constraints that have to be considered for the vitrector to operate efficiently. Because in many cases vitreous humor may need to be removed from the vicinity of the retina, the window in the casing needs to be as close as possible to the casing distal end. In addition, while in operation the needle distal tip oscillates across the casing window, the needle distal tip should not contact the closed casing distal end, since such contact leads to unacceptable levels of noise, as well as possibly damaging the needle. There is thus a small, but necessary, gap between the casing distal end and the most distal position of the needle distal tip.

Embodiments of the present disclosure provide a method for assembling a vitrector which addresses the constraints described above by enabling the needle distal tip to completely traverse the window, and provide the gap referred to above, without contacting the casing distal end.

The vitrector is formed from three sub-assemblies: an external needle casing, a distal housing portion, and a transmission unit. An assembly pin, which is not part of the vitrector, is used during assembly of the vitrector. The transmission unit includes the hollow needle, as well as an electric motor that oscillates the needle. In an initial assembly step, the transmission unit is inserted into the distal housing portion until it mates with a dedicated step in the distal housing portion. The needle distal tip of the unit is then positioned so that it is in its most extended state distally, and the assembly pin is inserted into the unit to fix the needle in the most distal position.

The external needle casing is mounted on a distal end of the distal housing portion and is fixedly attached to the distal housing portion, e.g., with glue.

Once the external needle casing has been fixedly attached to the distal housing portion, the assembly pin may be removed from the transmission unit. The removal enables the distal tip of the hollow needle to withdraw slightly from the internal surface of the casing, leaving a small gap between the internal surface and the needle distal tip. The small gap is present, even when, during operation of the vitrector, the needle is at its most distal position.

As explained above, the small gap permits operation of the vitrector without generating unacceptable noise levels, and without needle damage.

### System Description

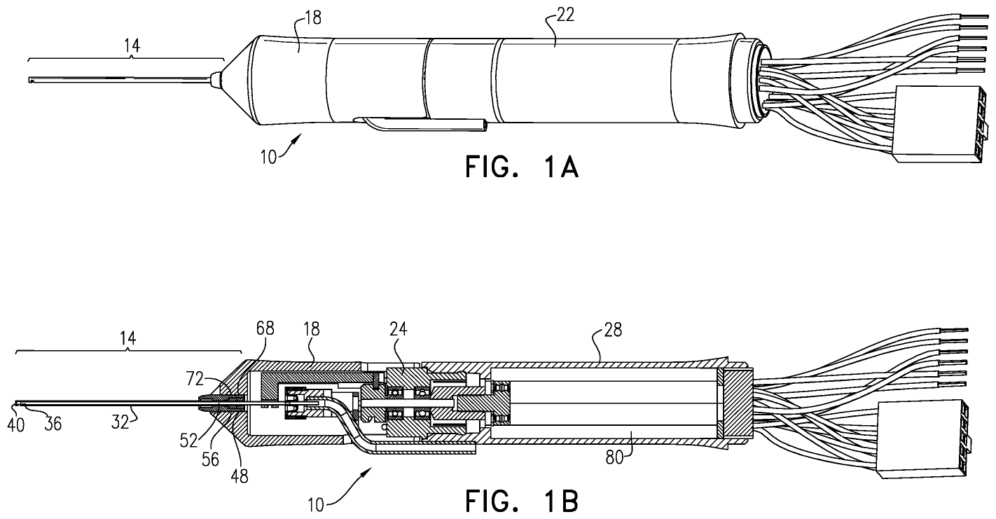

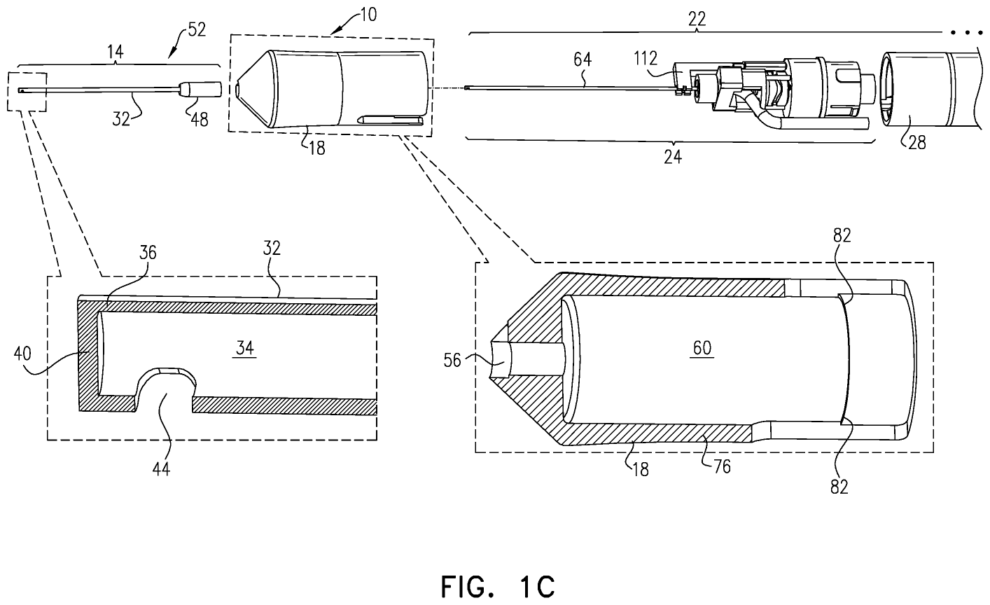

FIGS. 1A, 1B, and 1C are schematic diagrams of a vitrector 10. FIG. 1A is a perspective view, FIG. 1B is a cross-section, and FIG. 1C shows the vitrector divided into three sub-assemblies. The sub-assemblies are an external needle casing 14, a distal housing portion 18, and a transmission unit 22. Unit 22 is itself divided into two sections: a driving section 24 and a proximal housing portion 28 that is typically used as the handle of the vitrector. Proximal housing section 28 houses a motor 80. Distal housing portion 18 and proximal housing portion 28 together make up the vitrector housing that is grasped by the physician during its use.

External needle casing 14 comprises a tubular section 32 which is closed at a distal end 36 of the section by a termination 40. Within tubular section 32 is a lumen 34, and in the section, close to termination 40, there is an opening 44, also herein also termed a window 44. A casing coupling 48, located at a proximal end 52 of section 32 couples casing 14 to distal housing portion 18, and is configured to mate with a distal opening 56 in distal housing portion 18. Coupling 48 is configured, when mated with housing distal opening 56, to provide a sealed connection between lumen 34 and an internal lumen 60 of distal housing portion 18.

As is explained further below, a hollow tubular needle 64 of transmission unit 22 is positioned within lumen 34, and the needle is coaxially supported within the lumen by a spacer 68 and a sealing o-ring 72 residing within coupling 48, the spacer and the o-ring providing the sealed connection referred to above.

Distal housing portion 18 is a generally tubular element that tapers distally and includes housing distal opening 56. A wall 76 of distal housing portion 18 encloses internal housing lumen 60, and the wall has an annular step 82 formed therein (FIG. 1C).

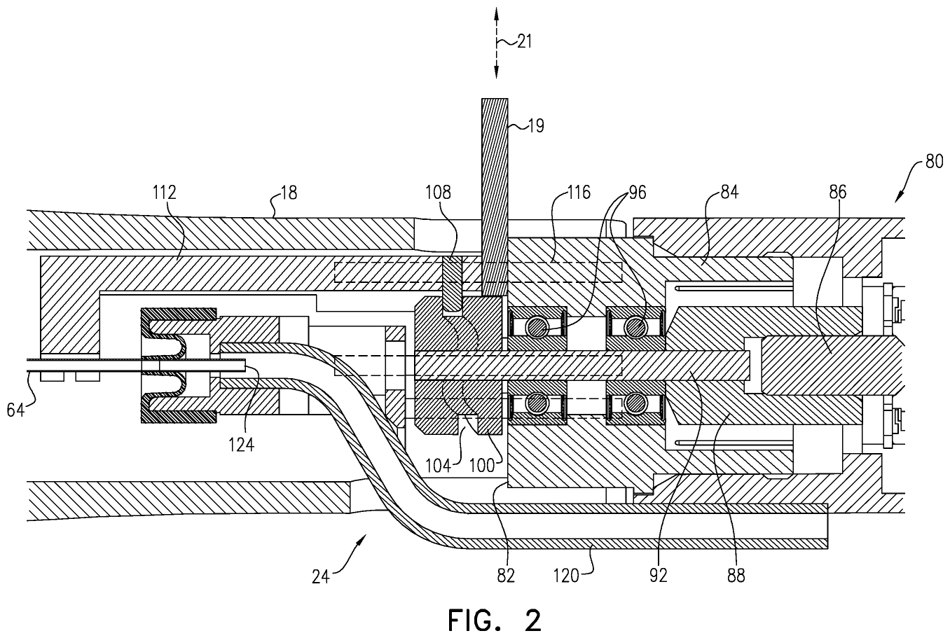

FIG. 2 is a cross-sectional view of driving section 24 mounted in distal housing portion 18. Driving section 24 is comprised of a motor 80 and a motor shaft coupling element 88 that couples a motor shaft 86 to a drive shaft 92. Drive shaft 92 is supported in a supporting structure 84 by bearings 96, and is connected to a cylindrical cam 100. There is an undulating groove 104 in an external radial surface of cam 100 and a cam follower 108, fixedly connected to a needle gripper 112 which fixedly grips needle 64, is constrained to travel in groove 104. Gripper 112 is itself constrained to only travel in a distal or proximal direction, parallel to drive shaft 92, by being configured to slide on rods 116—one rod 116 is visible in the cross-section. Rods 116 are parallel to drive shaft 92, are fixedly mounted into a bore within structure 84, and act as rails upon which gripper 112 can travel.

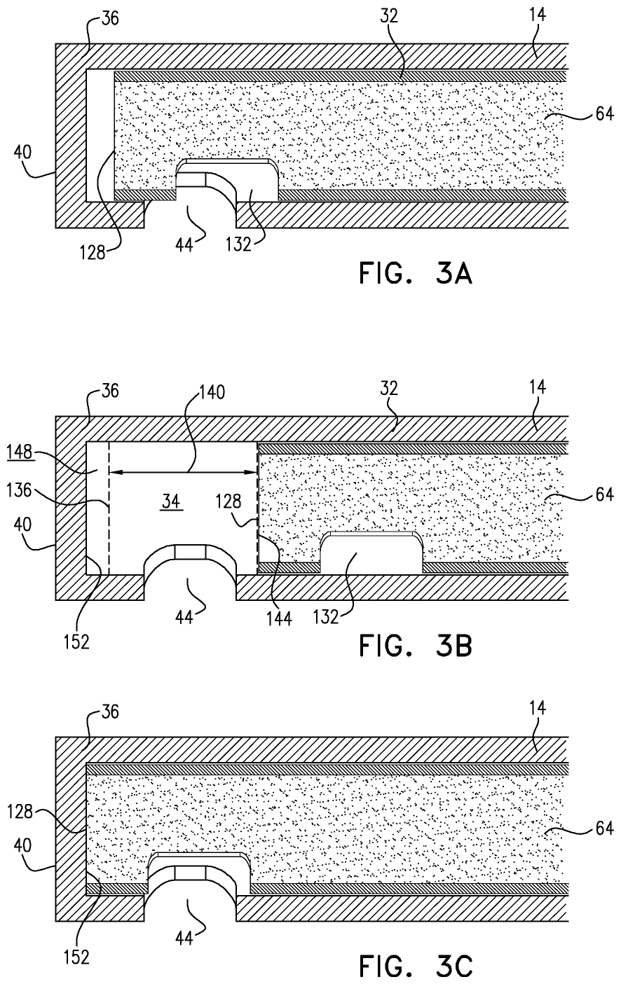

When motor 80 is powered on, it rotates drive shaft 92 and cam 100. The rotation of cam 100 imparts linear motion to follower 108 so that it oscillates distally and proximally, and, because follower 108 is fixed to gripper 112, the gripper and gripped needle 64 also oscillate distally and proximally. The amplitude of oscillation of needle 64 (also referred to below with reference to FIGS. 3A and 3B) corresponds to the amplitude of the change of undulating recess 104, measured parallel to shaft 92.

The needle oscillation cuts the vitreous humor, as described in more detail with respect to FIGS. 3A and 3B below. The cut material is aspirated by tubing 120, which is attached to a proximal end 124 of needle 64.

FIG. 2 also illustrates an assembly pin 19 which is used, as is described herein, during assembly of vitrector 10. During the assembly, pin 19 is removably inserted into distal housing portion 18 and driving section 24, the removable insertion being illustrated by double-headed arrows 21. Pin 19 is configured to fit between a distal side of structure 84 and a proximal side of gripper 112, and in an embodiment of the disclosure the fit is configured to be a tight, typically an interference, fit.

FIGS. 3A and 3B are schematic cross-sections respectively illustrating the most distal and most proximal positions of a distal tip 128 of needle 64, in casing 14, when vitrector 10 is in its assembled state, and when the needle is being oscillated by powering motor 80 on. It is the oscillation of distal tip 128 over window 44 that causes the cutting action of the vitreous humor. In some embodiments needle 64 has an aperture 132 formed in the needle, close to distal end 128, and the aperture enhances both the cutting action of the oscillation, and the aspiration of the cut material.

In FIG. 3B a broken line 136 shows the most distal position of distal tip 128, a double headed arrow illustrates an amplitude of oscillation 140 of the distal tip, and a broken line 144 shows the most proximal position of distal tip 128. It will be understood that amplitude of oscillation 140 of distal tip 128 has the same value as the amplitude of the change of undulating groove 104, measured parallel to shaft 92.

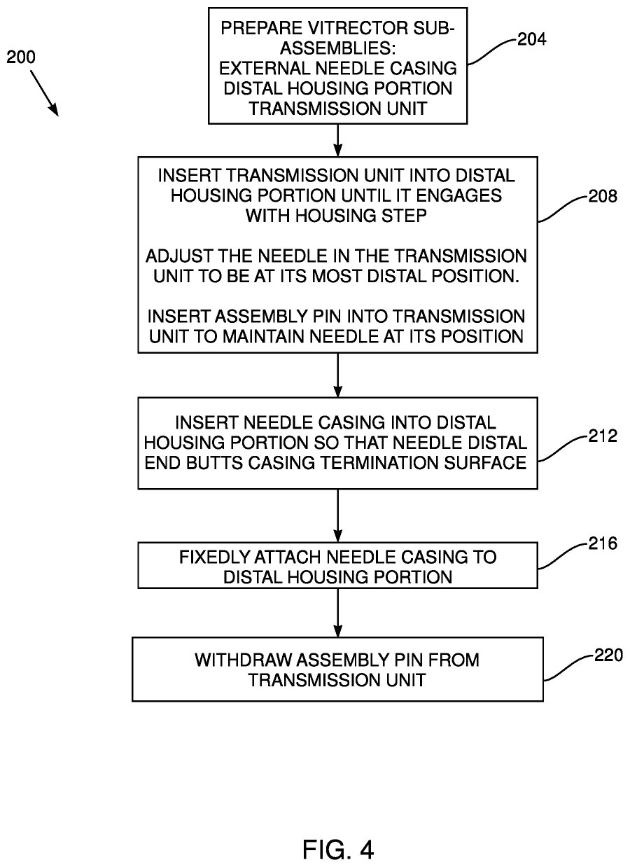

At the assembly of vitrector 10, described below with reference to the flowchart of FIG. 4, amplitude of oscillation 140 is configured to cover window 44. I.e., line 136 is distally beyond the distal end of the window, and line 144 is proximally beyond the proximal end of the window. As is also described below in the flowchart, a space 148 is configured between line 136, corresponding to the most distal position of needle distal tip 128, and an internal surface 152 of termination 40. Thus, the distance between surface 152 and line 136 is greater than zero.

FIG. 4 is a flowchart 200 of steps performed in assembling vitrector 10, and FIG. 3C is a schematic cross-section illustrating one of the steps.

In an initial step 204, the three sub-assemblies of the vitrector are prepared. I.e., coupling 48 is attached to tubular section 32, and spacer 68 and o-ring 72 are positioned within the coupling to form external needle casing 14. Distal housing portion 18 is a single generally tubular unit, having a wall 76 enclosing an internal lumen 60, as described above. Transmission unit 22 is formed of driving section 24 and handle 28. The elements of driving section 24, including, inter alia, motor 80 and cam 100, are described above with reference to FIG. 2.

In a needle positioning step 208, transmission unit 22 is inserted into distal housing portion 18 so that structure 84 of transmission unit 22 engages with step 82 of the housing. The engagement prevents motor 80, and any other parts of transmission unit 22, from moving further distally. At this stage transmission unit 22 may be fixed to the housing unit.

Cam 100 is then rotated, causing undulating groove 104 to rotate, so that distal tip 128 is moved to its most distal position. Assembly pin 19 is then inserted through distal housing potion 18 into transmission unit 22, between the distal side of structure 84 and the proximal side of gripper 112, as illustrated in FIG. 2. Because the fit of pin 19 is a tight fit, the elasticity of follower 108 causes the follower to bend slightly in a distal direction, so that distal tip 128 also moves slightly distally.

In a casing insertion step 212, needle casing 14 is mounted onto a distal end of distal housing portion 18, by inserting casing coupling 48 into distal opening 56 of the housing. The insertion comprises moving the casing proximally until, as shown in FIG. 3C, needle distal tip 128 butts internal surface 152 of termination 40.

Once casing 14 has been moved so that needle distal tip 128 butts internal surface 152 of termination 40, in a fixation step 216 the needle casing is fixed to distal housing portion 18, for example by gluing.

In a final step 220, assembly pin 19 is withdrawn from transmission unit 22. The withdrawal enables the elasticity of follower 108 to move gripper 112 and needle 64 proximally, so that space 148 (FIG. 3B) opens between the most distal position 136 of tip 128 and internal surface 152 of termination 40.

Once vitrector 10 has been assembled as described above, during operation of the vitrector there is a space, space 148, between the most distal position of the needle distal tip and the internal surface of the casing termination. During oscillation of the needle there is thus no contact between the needle distal end and the internal surface of the casing termination, but the needle distal tip completely traverses the window in the tubular section of the casing, thus ensuring there is efficient cutting and aspiration of the vitreous humor.

### EXAMPLES

Example 1. A method for assembling a vitrector (10), comprising:
providing a distal housing portion (18) for the vitrector, a transmission unit (22) configured to drive a needle (64) into oscillation, an assembly pin (19) configured to be inserted into the transmission unit so as to fix the needle in position, and a needle casing (14), having a lumen (34), the casing being configured to enclose the needle within the lumen;
fixing the transmission unit to the distal housing portion;
inserting the assembly pin into the transmission unit so as to fix a distal tip of the needle to be at a most distal position of the oscillation;
inserting the transmission unit, with the assembly pin inserted, to a predetermined position in the distal housing portion;
inserting the needle casing into the distal housing portion so that the casing encloses the needle and so that the distal tip butts a termination of the needle casing;
fixing the needle casing to the distal housing portion; and
removing the assembly pin from the transmission unit.

Example 2. The method according to example 1, wherein the predetermined position in the distal housing portion comprises a step therein.

Example 3. The method according to example 1, wherein the needle casing comprises a window therein.

Example 4. The method according to example 3, wherein the distal tip of the needle completely traverses the window during the oscillation.

Example 5. The method according to example 1, wherein the needle is hollow, and wherein the transmission unit comprises tubing, fixed to a proximal end of the needle, configured to aspirate material from the needle.

Example 6. The method according to example 1, wherein the transmission unit comprises a cylindrical cam having an undulating groove in an external cylindrical surface of the cam, the unit further comprising a cam follower coupled to the needle and constrained to travel in the groove so as to oscillate the needle on rotation of the cam.

Example 7. The method according to example 6, further comprising, prior to inserting the assembly pin into the transmission unit, rotating the cylindrical cam so that the distal tip of the needle is at the most distal position of the oscillation.

Example 8. A vitrector assembly, comprising:
a distal housing portion (18) for a vitrector (10);
a transmission unit (22) configured to drive a needle (64) into oscillation;
an assembly pin (19): and
a needle casing (14) having a lumen (34) configured to enclose the needle;
wherein:
the transmission unit is configured to be inserted to a predetermined position in the distal housing portion, and to be fixed therein,
the assembly pin is configured to be inserted into the transmission unit so as to fix a distal tip of the needle to be at a most distal position of the oscillation,
the needle casing is configured to be inserted into the distal housing portion so that the casing encloses the needle, so that the distal tip butts a termination of the needle casing, whereupon the needle casing is fixed to the distal housing portion, and
the assembly pin is configured to be removed from the transmission unit.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method for assembling a vitrector, comprising:
providing a distal housing portion for the vitrector, a transmission unit configured to drive a needle into oscillation, an assembly pin configured to be inserted into the transmission unit so as to fix the needle in position, and a needle casing, having a lumen, the casing being configured to enclose the needle within the lumen;
fixing the transmission unit to the distal housing portion;
inserting the assembly pin into the transmission unit so as to fix a distal tip of the needle to be at a most distal position of the oscillation;
inserting the transmission unit, with the assembly pin inserted, to a predetermined position in the distal housing portion;
inserting the needle casing into the distal housing portion so that the casing encloses the needle and so that the distal tip butts a termination of the needle casing;
fixing the needle casing to the distal housing portion; and
removing the assembly pin from the transmission unit.

2. The method according to claim 1, wherein the predetermined position in the distal housing portion comprises a step therein.

3. The method according to claim 1, wherein the needle casing comprises a window therein.

4. The method according to claim 3, wherein the distal tip of the needle completely traverses the window during the oscillation.

5. The method according to claim 1, wherein the needle is hollow, and wherein the transmission unit comprises tubing, fixed to a proximal end of the needle, configured to aspirate material from the needle.

6. The method according to claim 1, wherein the transmission unit comprises a cylindrical cam having an undulating groove in an external cylindrical surface of the cam, the unit further comprising a cam follower coupled to the needle and constrained to travel in the groove so as to oscillate the needle on rotation of the cam.

7. The method according to claim 6, further comprising, prior to inserting the assembly pin into the transmission unit, rotating the cylindrical cam so that the distal tip of the needle is at the most distal position of the oscillation.

8. A vitrector assembly, comprising:
a distal housing portion for a vitrector;
a transmission unit configured to drive a needle into oscillation;
an assembly pin: and
a needle casing having a lumen configured to enclose the needle;
wherein:
the transmission unit is configured to be inserted to a predetermined position in the distal housing portion, and to be fixed therein,
the assembly pin is configured to be inserted into the transmission unit so as to fix a distal tip of the needle to be at a most distal position of the oscillation,
the needle casing is configured to be inserted into the distal housing portion so that the casing encloses the needle, so that the distal tip butts a termination of the needle casing, whereupon the needle casing is fixed to the distal housing portion, and
the assembly pin is configured to be removed from the transmission unit.

9. The vitrector assembly according to claim 8, wherein the predetermined position in the distal housing portion comprises a step therein.

10. The vitrector assembly according to claim 8, wherein the needle casing comprises a window therein.

11. The vitrector assembly according to claim 10, wherein the distal tip of the needle completely traverses the window during the oscillation.

12. The vitrector assembly according to claim 8, wherein the needle is hollow, and wherein the transmission unit comprises tubing, fixed to a proximal end of the needle, configured to aspirate material from the needle.

13. The vitrector assembly according to claim 8, wherein the transmission unit comprises a cylindrical cam having an undulating groove in an external cylindrical surface of the cam, the unit further comprising a cam follower coupled to the needle and constrained to travel in the groove so as to oscillate the needle on rotation of the cam.

14. The vitrector assembly according to claim 13, further comprising, configuring, prior to inserting the assembly pin into the transmission unit, the cylindrical cam to rotate so that the distal tip of the needle is at the most distal position of the oscillation.
